(19) United States
(12) Patent Application Publication
(10) Pub. No.: US 2017/0274008 A1
(43) Pub. Date: Sep. 28, 2017

(54) Use of polysaccharide in Dendrobium officinale for anti-fatigue

(71) Applicant: Hong Kong Baptist University, Hong Kong (HK)

(72) Inventors: Quanbin HAN, Hong Kong (HK); Wei WEI, Hong Kong (HK)

(21) Appl. No.: 15/461,504

(22) Filed: Mar. 17, 2017

Related U.S. Application Data
(60) Provisional application No. 62/312,581, filed on Mar. 24, 2016.

(51) Int. Cl. A61K 31/736; A61K 9/00; A61K 36/8984

(52) U.S. Cl. CPC A61K 31/736; A61K 36/8984; A61K 9/0053

(57) ABSTRACT

The present invention relates to a glucomannan with a molecular size of 730 kDa, called DOP, which was previously identified as the unique authentication marker of the expensive herb Dendrobium officinals, as the main component for combating fatigue in subjects in need thereof. In particular, the present invention provides the required dosage for DOP to provide a potent anti-fatigue effect, stronger than Rhodiola rosea extract, and has significant potential to form an anti-fatigue health product.

### CROSS REFERENCE

This application is a non-provisional application of U.S. Provisional Patent Application Ser. No. 62/312,581 filed Mar. 24, 2016, which is hereby incorporated by reference in its entirety.

### FIELD OF INVENTION

The present invention relates to a glucomannan with a huge molecular size of 730 kDa, called DOP, which was previously identified as the unique authentication marker of the expensive herb Dendrobium officinale, as the main component for combating fatigue in subjects in need thereof. In particular, the present invention provides the required dosage for DOP to provide a potent anti-fatigue effect, stronger than Rhodiola rosea extract, and has significant potential to form an anti-fatigue health product.

### BACKGROUND OF INVENTION

Fatigue syndrome refers to difficulty in initiating or sustaining voluntary activities. It is a multifaceted illness because its pathophysiology and etiology are still unclear. Fatigue is often a combination of non-specific symptoms that accompany many diseases, such as aging, advanced cancer, depression, AIDS, multiple sclerosis, heart disease, diabetes, and Parkinson's disease. More than 24% of patients in primary-care clinics indicated that fatigue is a major problem. Fatigue syndrome is a serious worldwide prevalent health problem affecting over 800,000 American people and approximately 240,000 patients in the UK; 85% to 90% of these people are not receiving medical care that effectively reduces fatigue.

Another source of fatigue in modern populations is exercise. More and more people now exercise regularly to enhance their health. Excessive exercise also causes fatigue and even various types of damage to the body. Therefore, in the past few decades, health scholars and athletic physiologists have been looking for natural active compounds that can improve athletic ability, postpone fatigue, and accelerate the body's recovery from physical exertion. However, many of the active substances reported to address fatigue have side effects. For instance, Rhodiola L. extract of which salidroside is the main functional component, showed anti-fatigue effects, but excessive Rhodiola L. may result in hypoglycemia, which compromises recovery from fatigue. Therefore, safe and effective anti-fatigue natural products are still desired.

Tiepi Fengdou, the stem of Dendrobium officinale, has been used for thousands of years as a health tea herb in East Asia. It ranks as the first of “nine kinds of Chinese medicinal herbs” and is traditionally recorded as a tonic to nourish Yin, supply body fluids, strengthen immunity, and benefit gastric tonicity in traditional Chinese medicine theory. Despite a complicated chemical profile including bibenzyls, phenanthrenes, sesquiterpenoids, and other small compounds, polysaccharides are the dominant component of D. officinale, accounting for more than 50% of its total dry weight. The inventors' previous invention identified the unique authentication polysaccharide marker (glucomannan, called DOP) of this authentic Dendrobium species. DOP is likely also to be the main active ingredient because its content exceeds 30% of the dry herb by weight, and it shows immunomodulating effects towards immune cells.

It is the objective of the present invention to provide for a method of using DOP as anti-fatigue medicament.

Citation or identification of any reference in this section or any other section of this application shall not be construed as an admission that such reference is available as prior art for the present application.

### SUMMARY OF INVENTION

Accordingly, the first objective of the presently claimed invention relates to a glucomannan with a huge molecular size of 730 kDa, called DOP, which was previously identified as the unique authentication marker of the expensive herb Dendrobium officinale, as the main component for combating fatigue in subjects in need thereof. In particular, the present invention provides the required dosage for DOP to provide a potent anti-fatigue effect, stronger than Rhodiola rosea extract, and has significant potential to form an anti-fatigue health product.

In a first aspect of the present invention there is provided a method of alleviating body fatigue in a subject in need thereof by administrating a therapeutic amount of DOP extracted from the herb Dendrobium officinale.

In a first embodiment of the first aspect of the present invention there is provided a method of alleviating body fatigue in a subject in need thereof wherein the therapeutic amount of DOP is administered orally.

In a second embodiment of the first aspect of the present invention there is provided a method of alleviating body fatigue in a subject in need thereof wherein the DOP comprises a glucomannan with a molecular size of 730 kDa.

In a third embodiment of the first aspect of the present invention there is provided a method of alleviating body fatigue in a subject in need thereof wherein the therapeutic amount is at least 50 mg/kg administered on a daily basis.

In a fourth embodiment of the first aspect of the present invention there is provided a method of alleviating body fatigue in a subject in need thereof wherein the therapeutic amount is at least 4.27 mg/kg/day.

In a fifth embodiment of the first aspect of the present invention there is provided a method of alleviating body fatigue in a subject in need thereof wherein said subject is human.

In a second aspect of the present invention there is provided a use of DOP for the manufacture of a medicament for alleviating body fatigue in a subject in need thereof wherein said DOP comprises a glucomannan with a huge molecular size of 730 kDa extracted from the herb Dendrobium officinale.

In a first embodiment of the second aspect of the present invention there is provided a use of DOP for the manufacture of a medicament for alleviating body fatigue in a subject in need thereof wherein said medicament is administered to said subject via oral administration.

In a second embodiment of the second aspect of the present invention there is provided a use of DOP for the manufacture of a medicament for alleviating body fatigue in a subject in need thereof wherein said medicament is administered at a dosage of at least 50 mg/kg/day.

In a third embodiment of the second aspect of the present invention there is provided a use of DOP for the manufacture of a medicament for alleviating body fatigue in a subject in need thereof wherein said medicament is administered at a dosage of at least 4.27 mg/kg/day.

In a fourth embodiment of the second aspect of the present invention there is provided a use of DOP for the manufacture of a medicament for alleviating body fatigue in a subject in need thereof wherein said medicament is administered to human.

Throughout this specification, unless the context requires otherwise, the word “include” or “comprise” or variations such as “includes” or “comprises” or “comprising”, will be understood to imply the inclusion of a stated integer or group of integers but not the exclusion of any other integer or group of integers. It is also noted that in this disclosure and particularly in the claims and/or paragraphs, terms such as “included”, “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

Other aspects and advantages of the present invention will be apparent to those skilled in the art from a review of the ensuing description.

### BRIEF DESCRIPTION OF DRAWINGS

The above and other objects and features of the present invention will become apparent from the following description of the present invention, when taken in conjunction with the accompanying drawings, in which:

FIG. 1A shows Animal experimental design. Values are expressed as the mean±SD. Normal group means that mice are unexposed to weight-loaded swimming endurance test. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and PC group mice are all exposed to weight-loaded swimming test. *p<0.05, **p<0.01, compared with the control group.

FIG. 1B shows effects of DOP and Rhodiola extract in weight-loaded swimming endurance time. Values are expressed as the mean±SD. Normal group means that mice are unexposed to weight-loaded swimming endurance test. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and PC group mice are all exposed to weight-loaded swimming test. *p<0.05, **p<0.01, compared with the control group.

FIG. 2A shows the effects of DOP and Rhodiola extract on percentage of initial body weight. Values are expressed as the mean±SD. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and PC group mice are all exposed to weight-loaded swimming test. *p<0.05 compared with the control group.

FIG. 2B shows the effects of DOP and Rhodiola extract on initial food intake of BALB/c mice. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and PC group mice are all exposed to weight-loaded swimming test. *p<0.05 compared with the control group.

FIG. 3A shows the effects of DOP and Rhodiola extract on organ index: liver of BALB/c mice. Values are expressed as the mean±SD. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and the PC group mice are all exposed to weight-loaded swimming test. **p<0.01 compared with the control group.

FIG. 3B shows the effects of DOP and Rhodiola extract on organ index: kidney of BALB/c mice. Values are expressed as the mean±SD. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and the PC group mice are all exposed to weight-loaded swimming test. **p<0.01 compared with the control group.

FIG. 3C shows the effects of DOP and Rhodiola extract on organ index: heart of BALB/c mice. Values are expressed as the mean±SD. Control means that mice are given distilled water for 30 days. DOP means that mice are treated with DOP (50 mg/kg/day) for 30 days. PC means positive control, and mice of this group are treated with Rhodiola extract (100 mg/kg/day) for 30 days. The control group, the DOP group, and the PC group mice are all exposed to weight-loaded swimming test. **p<0.01 compared with the control group.

FIG. 3D shows the effects of DOP and Rhodiola extract on organ index: Spleen of BALB/c mice. Values are expressed as the mean±SD. Control means that mice are given distilled water for 30 days. DOP means that mice are treated with DOP (50 mg/kg) for 30 days. PC means positive control, and mice of this group are treated with Rhodiola extract (100 mg/kg) for 30 days. The control group, the DOP group, and the PC group mice are all exposed to weight-loaded swimming test. **p<0.01 compared with the control group.

FIG. 4A shows the effects of DOP and Rhodiola extract on serum biochemical parameters glutathione peroxidase (GSH-Px) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 4B shows the effects of DOP and Rhodiola extract on serum biochemical parameters superoxide dismutase (SOD) after weight-loaded swimming test. Values are expressed as the mean±SD (n=8). *p<0.05, **p<0.01, ***p<0.001, compared with the control group. Normal group means that mice are unexposed to the weight-loaded swimming test. The control group means that mice have been exposed to the weight loaded swimming test and treated with distilled water. PC means positive control, and mice of this group are treated with Rhodiola extract.

FIG. 4C shows the effects of DOP and Rhodiola extract on serum biochemical parameters blood urea nitrogen (BUN) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 4D shows the effects of DOP and Rhodiola extract on serum biochemical parameters lactic dehydrogenase (LDH) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 4E shows the effects of DOP and Rhodiola extract on serum biochemical parameters malondialdehyde (MDA) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 4F shows the effects of DOP and Rhodiola extract on serum biochemical parameters creatine phosphokinase CK) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 4G shows the effects of DOP and Rhodiola extract on serum biochemical parameters triglyceride (TG) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 4H shows the effects of DOP and Rhodiola extract on serum biochemical parameters lactic acid (LD) after weight-loaded swimming test. Values are expressed as the mean±SD (n=8). *p<0.05, **p<0.01, ***p<0.001, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 5A shows the effects of DOP and Rhodiola extract on liver glycogen after weight-loaded swimming test. Values are expressed as the mean±SD (n=8). *p<0.05, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 5B shows the effects of DOP and Rhodiola extract on gastrocnemius glycogen after weight-loaded swimming test. Values are expressed as the mean±SD (n=8). *p<0.05, compared with the control group. Normal group means that mice unexposed to the weight-loaded swimming test. The control group means that mice have been exposed to the weight loaded swimming test and treated with distilled water. PC means positive control, and mice of this group are treated with Rhodiola extract.

FIG. 6A shows the proliferation of lymphocytes of mice fed with DOP and Rhodiola rosea extract after stimulation by Lymphocytes (LPS). Lymphocytes (5×10⁵ cells/well) in 96-well-plate are incubated with LPS (20 μg/ml) for 48 hours. Lipopolysaccharides (LPS), a lipopolysaccharide which is a mitogen of B cells. The cell viability is measured with tetrazolium compound (MTS) method. The data are presented as the mean±SD. *p<0.05, ***p<0.001, compared with the control group. PC means positive control.

FIG. 6B shows the proliferation of lymphocytes of mice fed with DOP and Rhodiola rosea extract after stimulation by Concanavalin A (Con A). Lymphocytes (5×10⁵ cells/well) in 96-well-plate are incubated with Con A (2.5 μg/ml) for 48 hours. Concanavalin A (ConA), a phytohemagglutinin which is a mitogen of T cells. The cell viability is measured with tetrazolium compound (MTS) method. The data are presented as the mean±SD. *p<0.05, ***p<0.001, compared with the control group. PC means positive control.

FIG. 7 shows effects of DOP and Astragalus polysaccharide RAP. RAP is used as the positive control in weight-loaded swimming endurance time. Values are expressed as the mean±SD. Control means that mice are given distilled water for 30 days. DOP means that mice are treated with DOP (50 mg/kg/day) for 30 days. RAP means that mice are treated with Astragalus polysaccharide RAP (100 mg/kg/day) for 30 days. The control group, the DOP group, and RAP group mice are all exposed to weight-loaded swimming test. *p<0.05, **p<0.01, compared with the control group.

### DETAILED DESCRIPTION OF THE INVENTION

The present invention is further illustrated by the following experiments or embodiments by which it should be understood that the subject matter disclosed in the experiments or embodiments may only be for illustrative purpose but is not intended to limit the scope of the claimed invention:

The inventors' previous invention, described in U.S. Pat. No. 8,999,719, the disclosure of which is incorporated by reference herein, identified the unique authentication polysaccharide marker (glucomannan, called DOP) of this authentic Dendrobium species. In the present invention DOP was determined to be the main active ingredient because its content exceeds 30% of the dry herb by weight, and it shows immunomodulating effects towards immune cells. Therefore, the inventors hypothesized that DOP also has anti-fatigue activity. In the present invention, the anti-fatigue activity of DOP is evaluated using BALB/c mice in a repeated weight-loaded endurance swimming test. The contents of lactic dehydrogenase (LDH), creatine phosphokinase (CK), triglyceride (TG), blood urea nitrogen (BUN), superoxide dismutase (SOD), malondialdehyde (MDA), lactic acid (LD), and glutathione peroxidase (GSH-Px) in serum, the glycogen of liver and gastrocnemius muscle, and the cell variability of T lymphocytes and B lymphocytes are also determined to clarify the underlying mechanism of action.

Materials and Methods

Materials

Rhodiola rosea extract containing 1% salidroside is purchased from Fleurance Nature, France, and used as the positive control. Triglyceride assay kits, lactic dehydrogenase assay kits, malonaldehyde assay kits, superoxide dismutase assay kits, glutathione peroxidase assay kits, lactic acid assay kits, urinary nitrogen assay kits, hepatic glycogen/muscle glycogen assay kits, creatine kinase assay kits are all purchased from Nanjing Jiancheng Bioengineering Institute (Jiangsu, China). Lipopolysaccharides (LPS, from Escherichia coli 0111:B4) and Concanavalin A (Con A) are purchased from Sigma-Aldrich (St. Louis, Mo., USA). CellTiter 96® AQueous One Solution Cell Proliferation kit is purchased from Promega Inc. (Madison, Wis., USA). The authentic Dendrobium officinale sample, also named Tiepi Fengdou, is provided by a certified production area in mainland China and authenticated by Dr. Chen Hubiao. Voucher specimens are deposited at the School of Chinese Medicine in Hong Kong Baptist University, Hong Kong. Polysaccharide marker of Dendrobium officinale (DOP) is prepared in the inventors' previous invention, cited above.

Animals and Experimental Design

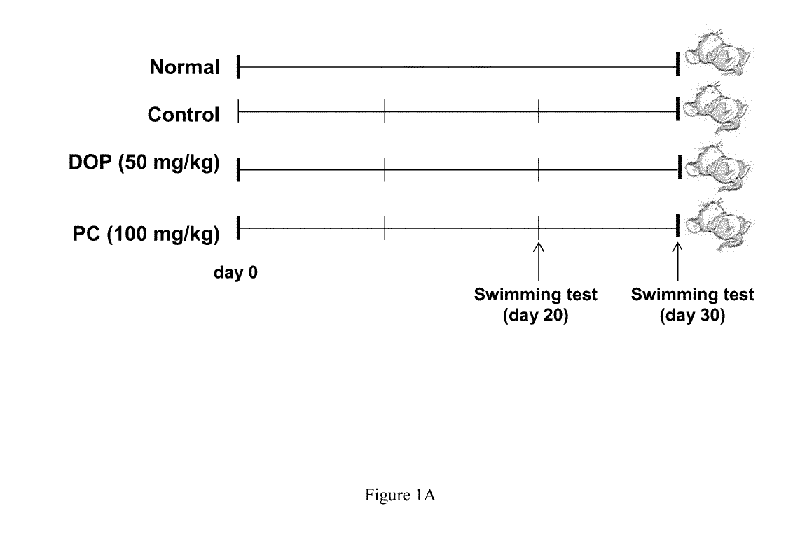

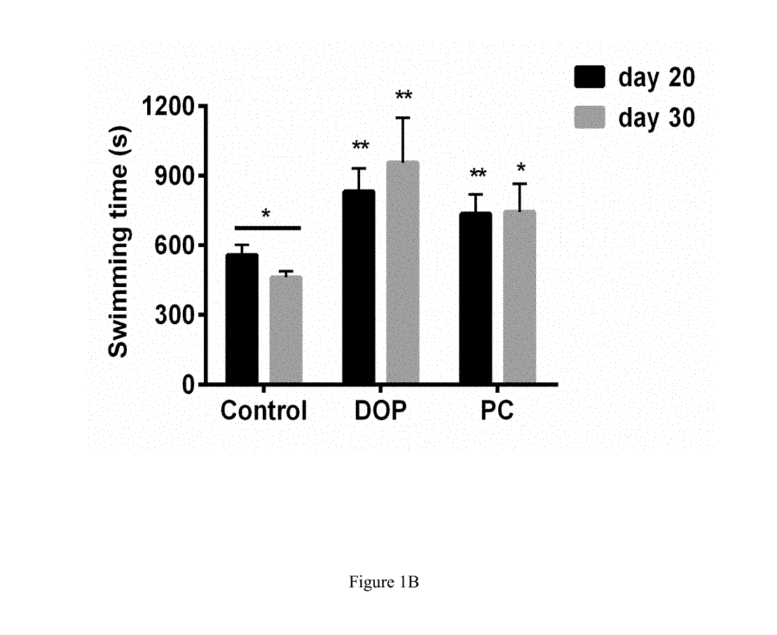

Inbred strain male (6 to 8 week-old, 22±2 g) BALB/c mice are purchased from the Laboratory Animal Services Centre of The University of Hong Kong. The animals are provided with standard pellet diet and water ad libitum and maintained under controlled conditions of temperature and humidity, with a 12 hours light/dark cycles. All experiments with animals are carried out in accordance with the Animals Ordinance, Department of Health, Hong Kong Special Administration Region, China for the care and use of experimental animals. All of the experimental protocols are first approved by the Committee on Use of Human and Animal Subjects in Teaching and Research of the Hong Kong Baptist University. The animals are used for experiments after 7 days of adaptation to the environment and the standard diet. Mice are trained to accustom themselves to swimming twice (10 min per time) in the first week. Mice which could not learn to swim are screened out. As shown in FIG. 1A, trained mice are randomly divided into four groups, each consisting of 8 mice.

Group 1 (Normal). Mice do not receive any treatment.

Group 2 (Control). Mice are given distilled water for 30 days.

Group 3 (DOP). In this example, the average body weight of mice is 22 g; and the mice are treated with DOP (50 mg/kg/day) for 30 days.

According to the commonly used Meeh-Rubner conversion formula, the dose ratio between mice and human is approximately 12:1, so the human doses of DOP will be at least 4.27 mg/kg/day.

In details:

Human dose (mg/kg)=mice dose (mg/m²)×human BSA (m²)/human body weight (kg)

Human BSA=0.0061×body height (cm)+0.0128×body weight (kg)−0.1529;

Mice dose (mg/m²)=mice dose (mg)/mice BSA (m²);

Mice BSA=9.1×(W^{2/3}/10000).

W represents body weight (g), BSA represents body surface area.

Suppose the average human body weight is 60 kg, the height is 170 cm, so:

Human BSA=0.0061×170+0.0128×60−0.1529=1.6521 m²

Mice BSA=9.1×22^{2/3}/10000=0.0071 m²

Mice dose (mg/m²) of the DOP=50×22×10⁻³/0.0071=154.9 mg/m²

Therefore,

Human dose (mg/kg) of the DOP will be 154.9×1.6521/60=4.27 mg/kg/day

Group 4 (PC). Mice are treated with Rhodiola rosea extract (100 mg/kg/day) for 30 days, as a positive control. These doses correspond to a typical human dose of 600 mg given to a 60 kg person (applying the coefficient equal to 10 for adjusting for differences between mouse and human in relation of the surface to body mass).

DOP and Rhodiola rosea extract is dissolved in distilled water and fed by gavage to mice once a day. Changes in the body weight of the mice are observed every seven days. The above method of grouping and feeding is repeated to determine related indicators.

Mice are anesthetized with chloral hydrate and blood samples are collected from each treatment group. Serum samples are obtained by centrifugation (3000 rpm, 10 min, 4° C.) and stored at −80° C. for further analysis. The spleens, hearts, and livers are weighed and their weights relative to the final body weights (organ index) are calculated.

Weight-Loaded Swimming Endurance Time

Briefly, 1 hour after the last oral administration, mice are placed in the swimming pool (50 cm×50 cm×40 cm) filled with fresh water at 25±1° C., approximately 30 cm deep so that mice could not support themselves by touching the bottom with their feet. A tin wire (5% of body weight) is loaded on the tail root of the mouse. It is reported that this arrangement forces the mouse to maintain continuous rapid leg movement. The swimming period is regarded as the time spent by the mouse floating in the water, struggling until exhausted. The mice are assessed to be exhausted when they fail to rise to the surface of water to breathe within a 10 seconds period. At the end of the session, the mice are removed from the water, dried with paper towels, and placed back in their home cages.

Biochemical Analysis

After 28 days, the mice are taken out from each group for analyses of hepatic glycogen, muscle glycogen, and blood biochemical parameters. One hour after the last intragastric administration of DOP and Rhodiola rosea extract, the mice are forced to swim in the swimming pool (weight-loaded) for 6 minutes session according to the method in Materials and Methods. At the end of the session, mice are removed from the water, dried with a paper towel and anesthetized with intraperitoneal injection of chloral hydrate. After anesthetization, blood is collected in heparinized tubes and tubes without anticoagulant by removing the left eyeball. Serum is prepared by centrifugation at 3500 rpm at 4° C. for 15 min. The blood plasma is tested to determine the concentration of lactic dehydrogenase (LDH), creatine phosphokinase (CK), triglyceride (TG), blood urea nitrogen (BUN), superoxide dismutase (SOD), malondialdehyde (MDA), lactic acid (LD), and glutathione peroxidase (GSH-Px) using commercial kits as listed in Materials and Method.

Analysis of Tissue Glycogen Contents

After the blood was collected, the livers and the gastrocnemius muscle of the mice are immediately dissected, frozen in liquid nitrogen, and kept at −80° C. until analysis of glycogen concentration. The concentration of hepatic glycogen is tested following the recommended procedures provided by the hepatic glycogen/muscle glycogen assay kits. Briefly, alkaline solutions are added to liver and gastrocnemius muscle samples for hydrolysis at 100° C. for 30 min. After centrifugation at 4000×g for 15 minutes, the supernatants are discarded. 0.5 ml of distilled water and 1 ml of 0.2% anthrone are added, and the vials are placed in a boiling-water bath for 20 min. The absorbance at 620 nm of the solution in vials is determined by spectrophotometer (Bio-Rad, Richmond, Calif., USA).

Lymphocyte Proliferation Assays

Spleens are collected from BALB/c mice of each group after killing them by cervical dislocation. Single cell suspension of splenocytes is prepared according to the method descried by Busse (Busse, C. E., Czogiel, I., Braun, P., Arndt, P. F., Wardemann, H., 2014. Single-cell based high-throughput sequencing of full-length immunoglobulin heavy and light chain genes. European journal of immunology 44, 597-603.). Briefly, the spleens are cut into several pieces and pressed through a 70 μm cell strainer (BD falcon, BD Biosciences, San Jose, Calif.) into culture medium using a syringe plunger. Spleen cells are re-suspended in red cell lysis buffer and incubated at room temperature for 5 minutes. The resulting pellet is re-suspended and diluted to 5×10⁶ cells/ml with RPMI-1640 after the cell viability is assessed by trypan blue exclusion. The 100 μl cell suspension is incubated in 96-well culture plates. It is known that LPS and Con A stimulate B cells and T cells, respectively. Thus, the lymphocyte proliferation is tested by incubating the mouse lymphocytes in the absence or presence of LPS and Con A at the optimal concentration (LPS: 20 μg/ml and Con A: 2.5 μg/ml) for 48 hours. After that, 20 μl of CellTiter 96® AQueous One Solution Cell Proliferation reagent is added into each well at 4 hours before the end of incubation. The absorbance of cells in each well is measured by Benchmark Plus microplate reader (Bio-Rad, Richmond, Calif.) at a wave length of 490 nm.

Statistical Analysis

All values are expressed as means±standard error (S.E.) in the tables and are indicated by vertical bars in the figures. Data are analyzed by one-way ANOVA, and then differences among means are analyzed using Fisher's protected least significant differences (LSD) multi-comparison test. Differences are considered significant at p<0.05.

Results

Weight-Loaded and Forced Swimming Endurance Test in Mice

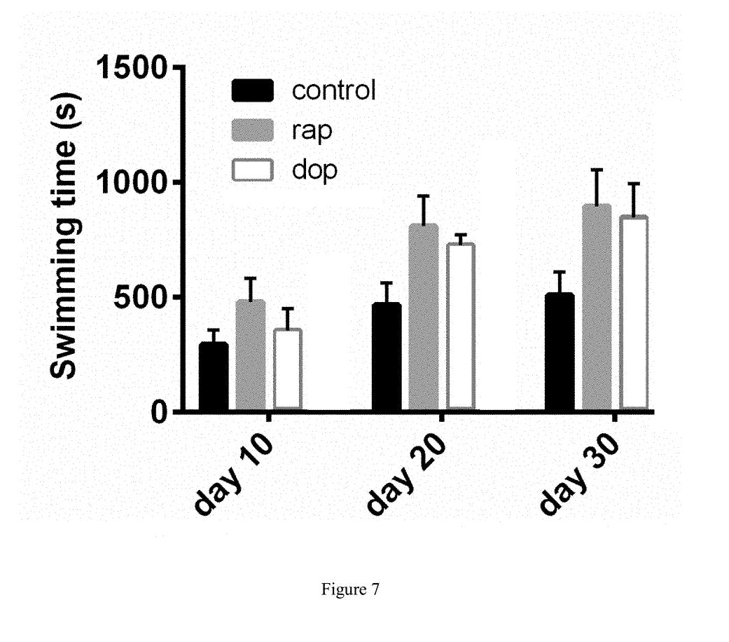

The weight-loaded and forced swimming endurance test, one of the commonly-used anti-fatigue test models, is chosen to evaluate the anti-fatigue effect of DOP. The duration of mean exhausting swimming time indicates the degree of anti-fatigue. As shown in FIG. 1B, the positive control (PC) Rhodiola extract significantly increases the swimming time (736.5±81.08 seconds, p<0.01) in comparison with the control group (557±45.42 seconds) at day 20. And the DOP group exhibited an even longer swimming time around 832.33 seconds. It is suggested that DOP's anti-fatigue effect may be stronger than the positive control. Different from the normal anti-fatigue test, these three groups continued to be fed for another 10 days. At day 30, the second swimming test is performed. The control group obviously do not recover from fatigue because the swimming time significantly decreases to (461.33±22.23 seconds, p<0.05), compared to the first time. The PC group does not show such a decrease and remain at the same level. Strikingly, the swimming time of the DOP group continues to increase to around 956.75 seconds. These results suggests that DOP is a better anti-fatigue substance than Rhodiola extract. In FIG. 7, the swimming time of the DOP group is compared to a group treated with Astragalus polysaccharide RAP. As the results showed, both DOP and RAP showed significant ability in prolonging the swimming time of the tested mice.

Effects of DOP and Rhodiola Extract on Body Weight and Organ Index

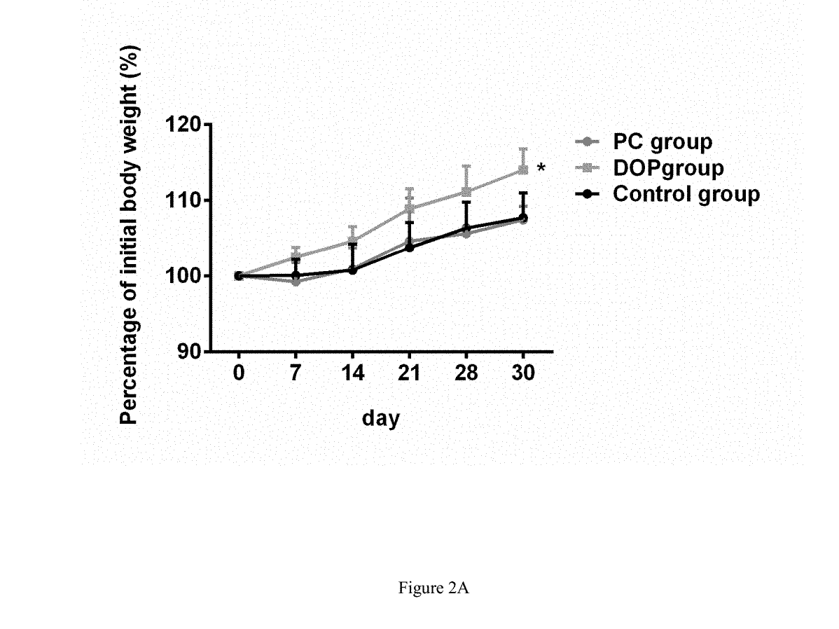

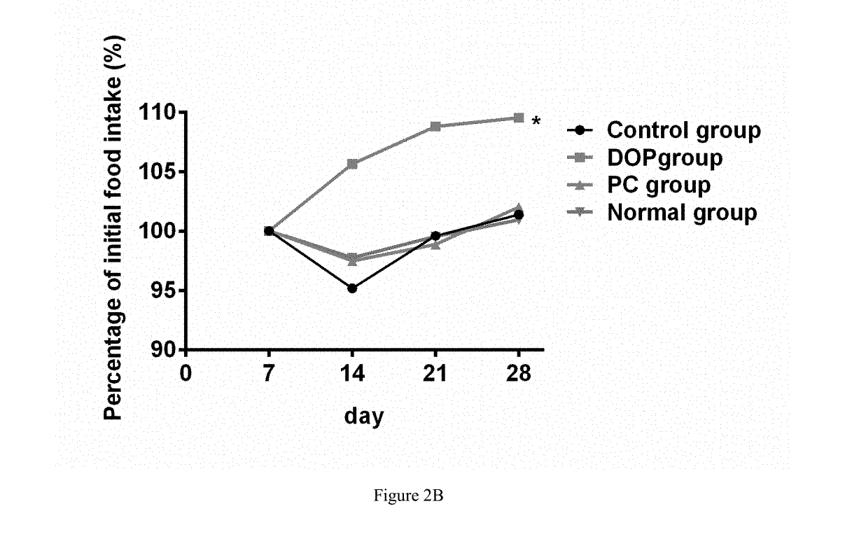

As shown in FIG. 2A and Table 1, compared to body weights on the first day, the body weight of the control group, the DOP group, and Rhodiola extract group all increase during the experiment. When comparing to the control group and PC group, DOP has a significant effect on body weight gain (p<0.05). Consistently, the food consumption rate (Table 2) of the DOP group increases significantly during the course of the experiment when compared to the food consumption rate of the control group, positive group, and normal group (FIG. 2B, p<0.05).

TABLE 1
Effects of DOP and Rhodiola extract on body weight (g) of BALB/c mice.

| | Day 1 | Day 7 | Day 14 | Day 21 | Day 28 |
|---|---|---|---|---|---|
| Normal goup | 21.54 ± 0.52 | 22.18 ± 0.52 | 22.74 ± 0.72 | 23.28 ± 1.05 | 24.08 ± 0.77 |
| Control group | 22.53 ± 0.43 | 22.54 ± 0.63 | 22.70 ± 0.98 | 23.68 ± 0.77 | 23.96 ± 0.91 |
| DOP group | 22.42 ± 1.02 | 22.84 ± 0.93 | 23.16 ± 1.29 | 24.10 ± 1.43 | 24.59 ± 1.54 |
| PC group | 23.38 ± 0.33 | 23.21 ± 0.72 | 23.61 ± 0.87 | 24.44 ± 1.17 | 24.76 ± 0.38 |

The data are presented as the mean ± SD (n = 8).
Normal group means mice are unexposed to weight-loaded swimming endurance test.
The control group means mice are exposed to weight-loaded swimming endurance test and treated with distilled water. PC group means positive group, Rhodiola extract group.

TABLE 2
Effects of DOP and Rhodiola extract on food intake (g) of BALB/c mice.

| | First week | Second week | Third week | Fourth week |
|---|---|---|---|---|
| Normal group | 186.3 | 182.1 | 185.4 | 188.1 |
| The control | 194.8 | 185.4 | 194.0 | 197.5 |
| group | | | | |
| The DOP group | 203.3 | 214.8 | 221.2 | 222.7 |
| PC group | 202.6 | 197.5 | 200.3 | 206.7 |

Normal group means mice are unexposed to weight-loaded swimming endurance test.
The control group means mice are exposed to weight-loaded swimming endurance test and treated with distilled water. PC group means positive group, Rhodiola extract group.

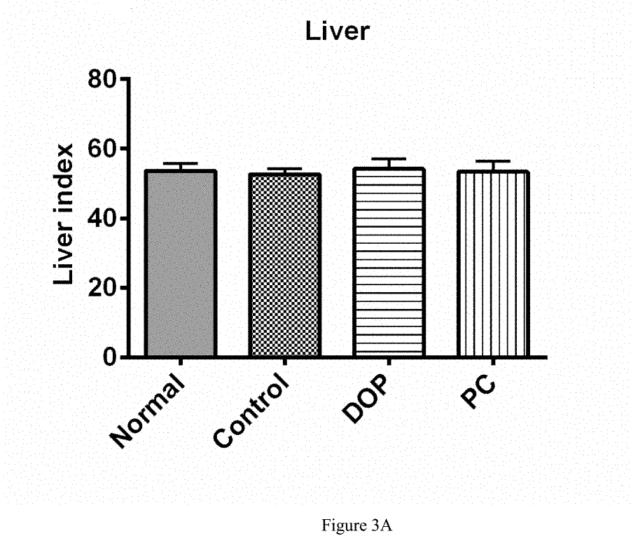

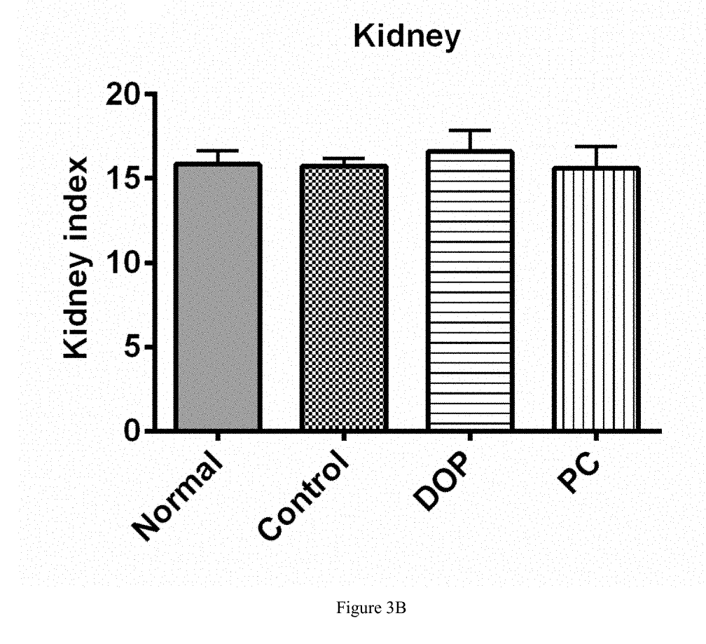

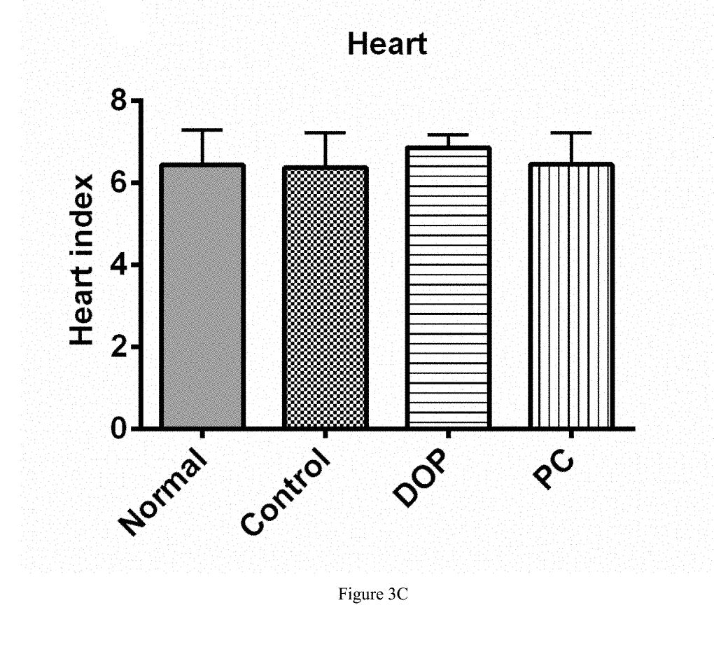

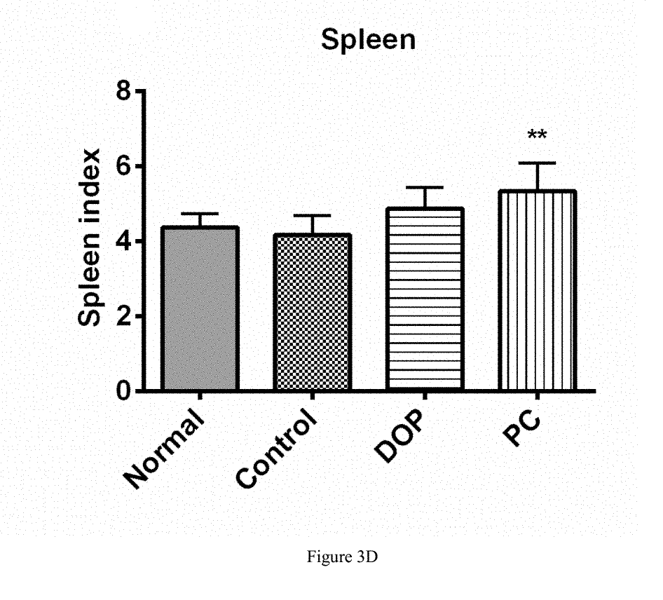

The organ indexes of liver, heart, kidney, and spleen is further evaluated. The results are presented in Table 3 and FIG. 3. Oral administration of DOP and Rhodiola extract at 50 mg/kg/day and 100 mg/kg/day, respectively, for 4 weeks and swimming test slightly, but not significantly, ameliorated the organ index of (FIG. 3C) heart, (FIG. 3A) liver, (FIG. 3B) kidney. As shown in FIG. 3D, the spleen index of the control group decreases slightly after weight-loaded swimming test compared to that of the normal group, but spleen index of the DOP group is increased slightly comparing to that of the control group. Strikingly, Rhodiola extract remarkably increases the organ index of spleen in comparison with that of the control group (FIG. 3D, p<0.01).

TABLE 3
Effects of DOP and Rhodiola extract on organ index of BALB/c mice.

| | liver | heart | kidney | spleen |
|---|---|---|---|---|
| Normal group | 53.70 ± 1.90 | 6.43 ± 0.85 | 15.84 ± 0.73 | 4.37 ± 0.32 |
| The control | 52.73 ± 1.30 | 6.36 ± 0.73 | 15.75 ± 0.43 | 4.17 ± 0.47 |
| group | | | | |
| The DOP | 54.25 ± 2.52 | 6.86 ± 0.27 | 16.60 ± 1.18 | 4.86 ± 0.49 |
| group | | | | |
| PC group | 53.44 ± 2.62 | 6.44 ± 0.70 | 15.60 ± 1.17 | 5.34 ± 0.68 |

The data were presented as means ± S.D. (n = 8).
Organ index = weight of organ (mg)/body weight (g).
PC group means positive group, Rhodiola extract group.

Effects of DOP and Rhodiola Extract on Serum Biochemical Parameters

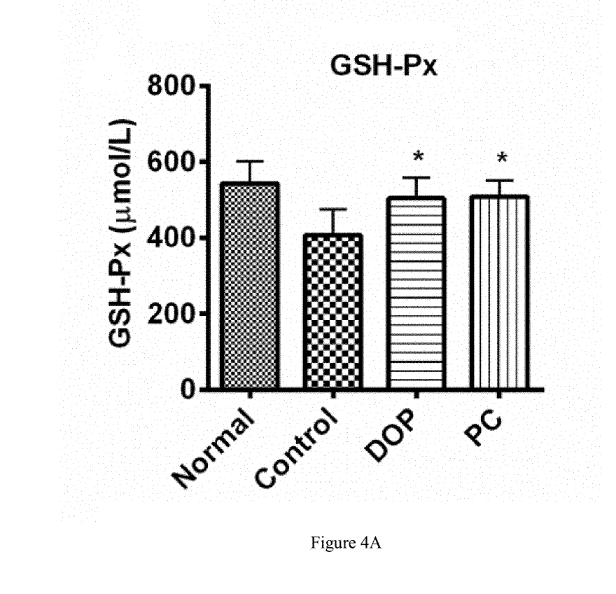

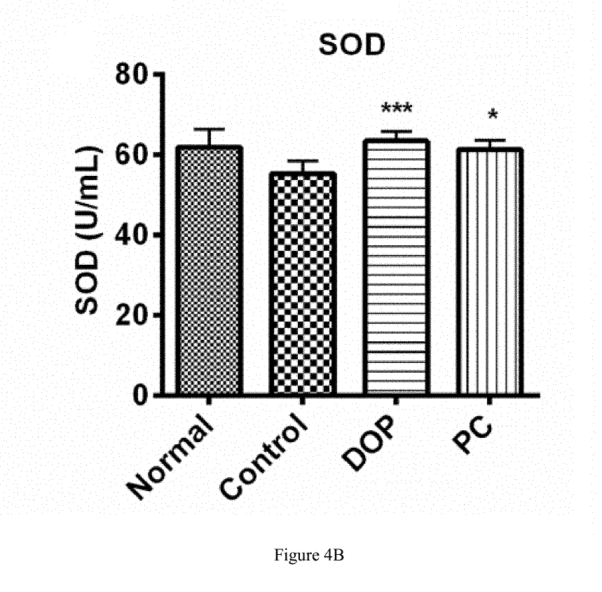

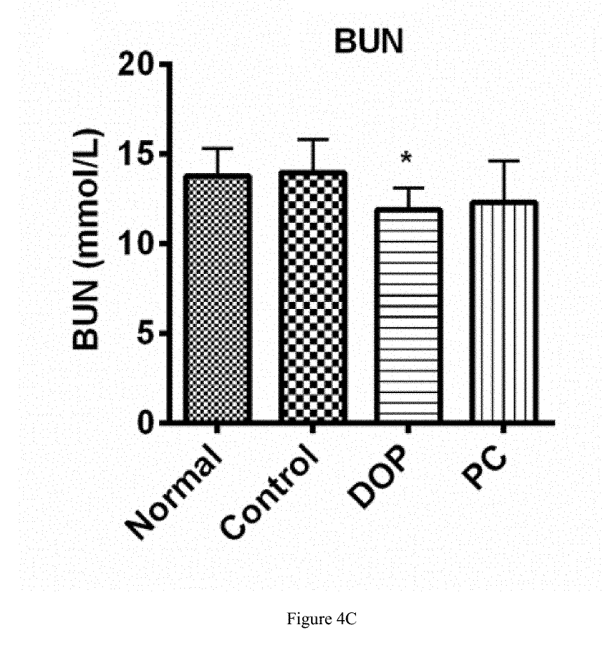

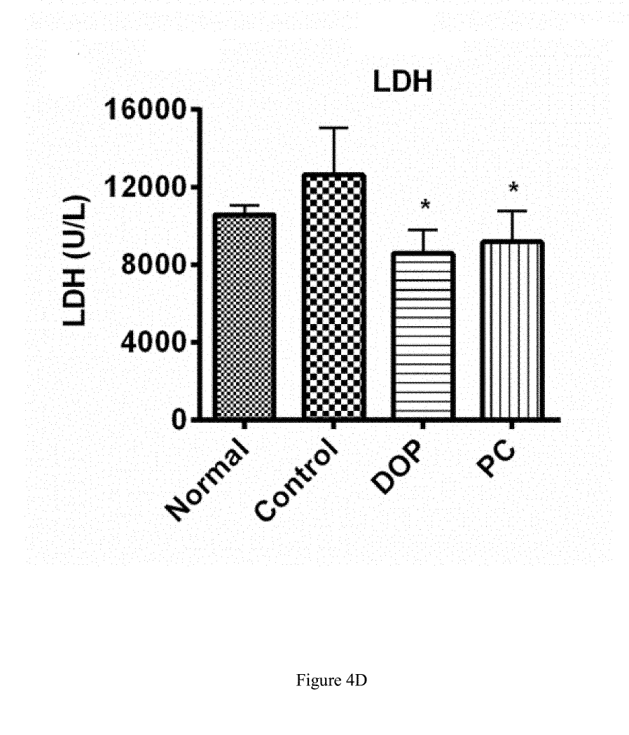

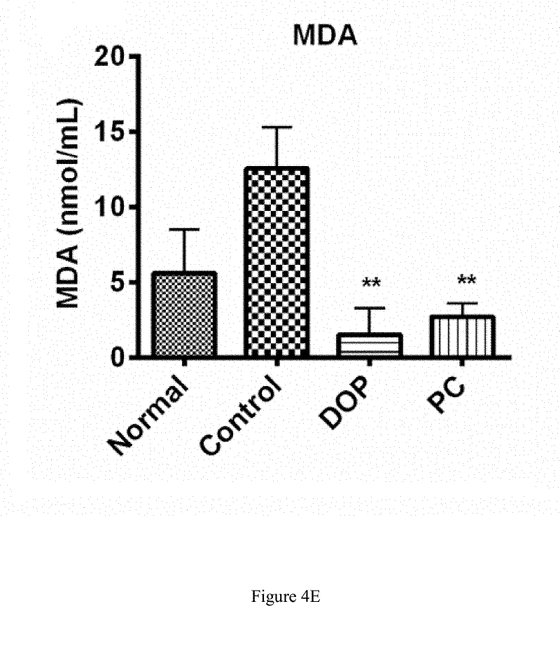

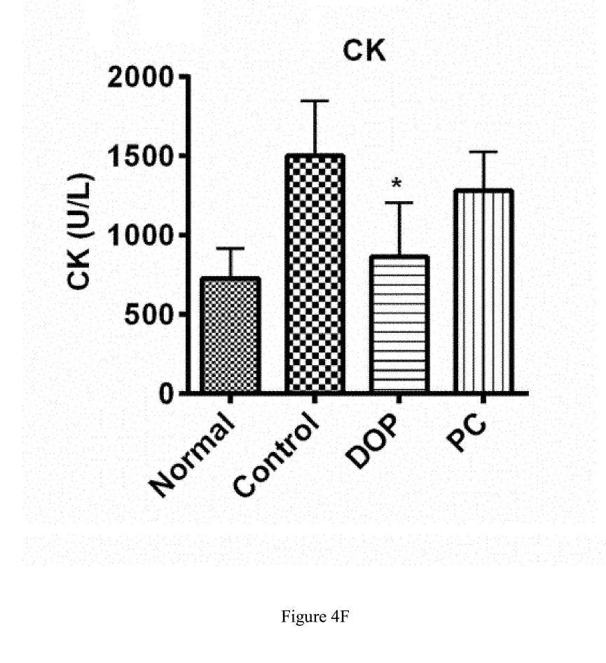

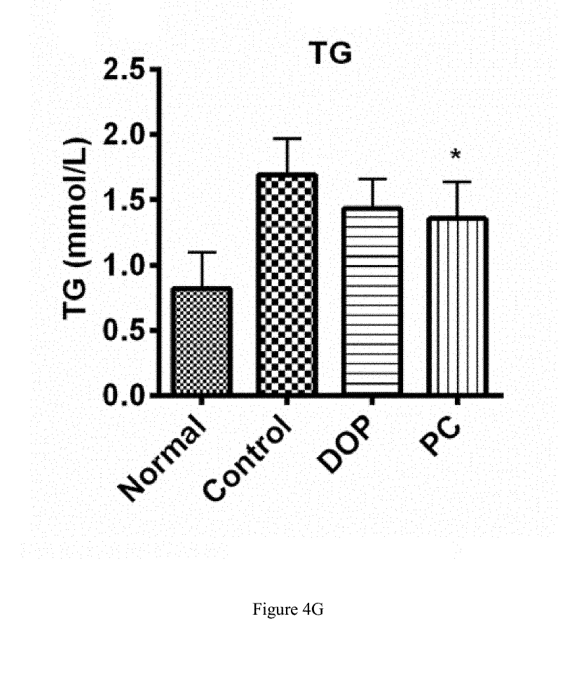

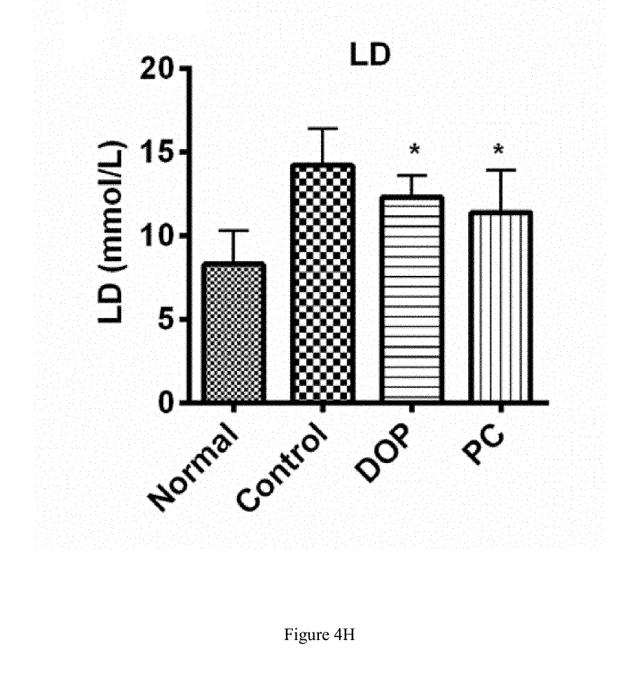

Blood biochemical parameters are determined to clarify the anti-fatigue mechanism. As shown in FIG. 4, the weight-loaded and forced swimming test induces an increase of (FIG. 4C) BUN, (FIG. 4D) LDH, (FIG. 4E) MDA, (FIG. 4F) CK, (FIG. 4G) TG and (FIG. 4H) LD levels in serum of mice in the control group, comparing to the normal group. These effects are partially attenuated by DOP and Rhodiola extract. By contrast, exposure to the forced swimming test led to a decrease in (FIG. 4B) SOD and (FIG. 4A) GSH-Px levels of the control group and all these effects are blocked by DOP and Rhodiola extract.

Effects of DOP and Rhodiola Extract on Glycogen in Liver and Gastrocnemius

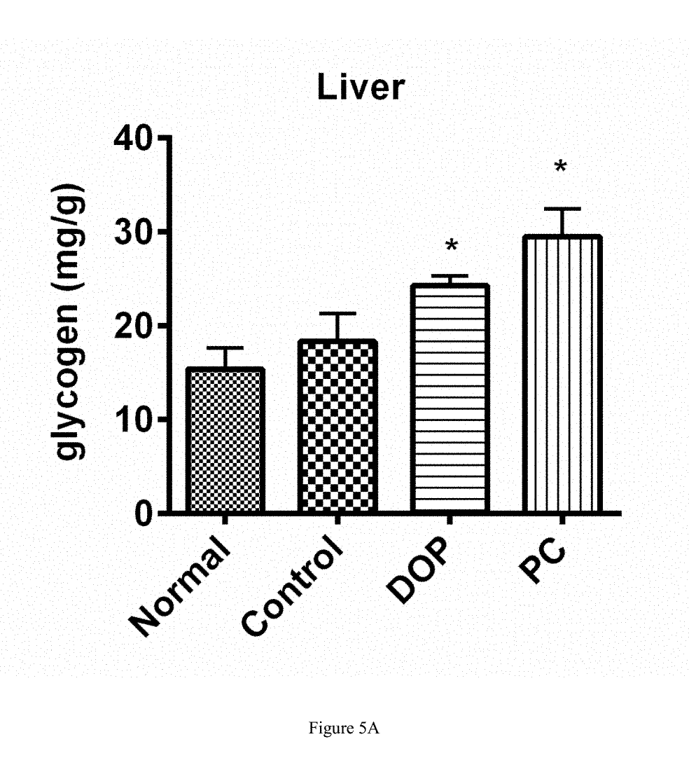

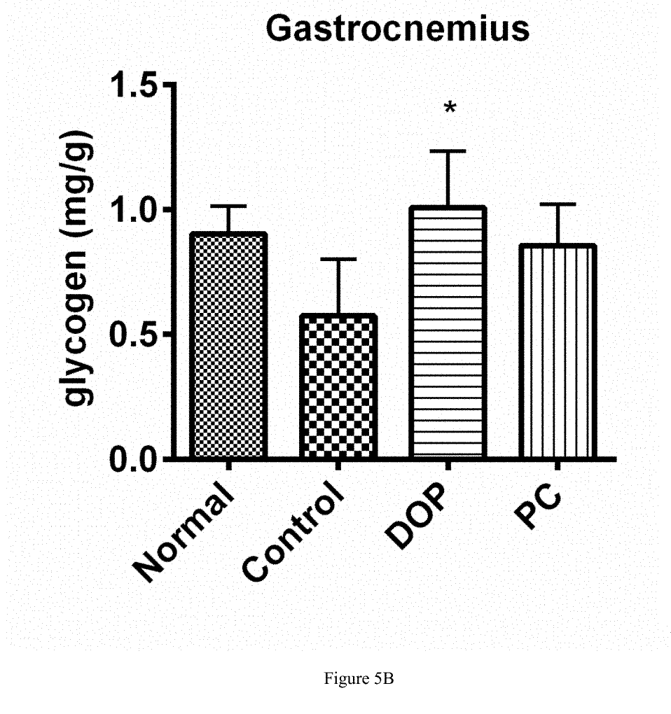

Glycogen in liver and gastrocnemius are determined by hepatic glycogen/muscle glycogen assay kits. As shown in FIG. 5, the storage of hepatic glycogen increases after swimming test. Simultaneously, DOP and Rhodiola extract enhances the hepatic glycogen level in mice significantly comparing to that of the control group (FIG. 5A, p<0.05). DOP also boosts glycogen in the gastrocnemius muscle of mice significantly comparing to that of the control group (FIG. 5B, p<0.05). In contrast, Rhodiola extract does not significantly increase the glycogen in the gastrocnemius of mice in the invention.

DOP's Effect on Proliferation of Mouse Lymphocytes

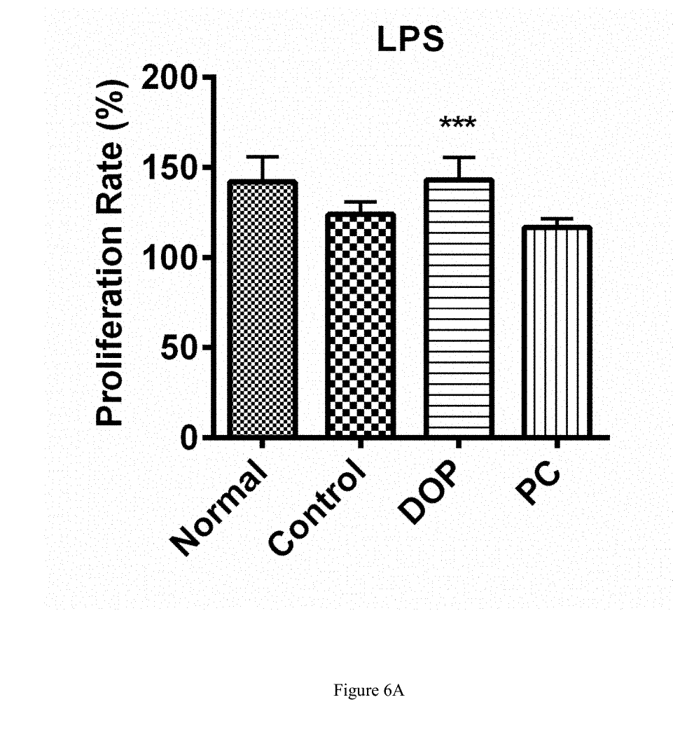

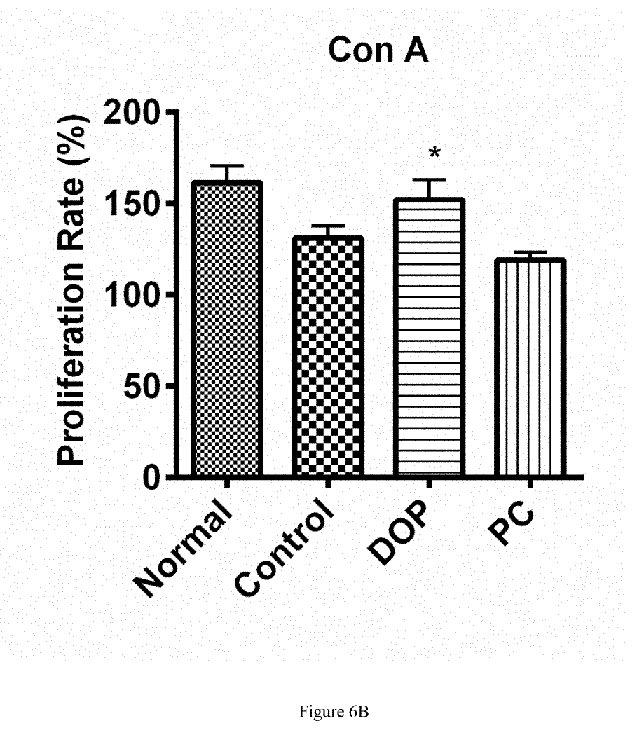

After feeding mice with DOP and Rhodiola extract for 30 days, the lymphocytes from spleens of each group is subjected to a lymphocyte proliferation assay to assess the physical immunity. As shown in FIG. 6, a significant increase of proliferation rates of lymphocytes is stimulated by FIG. 6A LPS and FIG. 6B Con A is observed in the DOP groups (p<0.05), but it is not detected in the positive control group (Rhodiola extract), comparing to the control group.

Discussion

The present invention evaluates the anti-fatigue effects and underlying mechanism of DOP and Rhodiola extract in mice. DOP and Rhodiola extract extend the weight-loaded swimming time and facilitate oxidative enzyme activity, storage of hepatic glycogen and responses of T cells to mitogens, suggesting that both DOP and Rhodiola extract contributes to enhancement of physical strength and endurance.

Many Chinese research groups have demonstrated that D. officinale, D. officinale health tea and compounds containing D. officinale have anti-fatigue and immunomodulating effects. However, they do not find which phytochemical component of D. officinale is responsible for this anti-fatigue activity. The results of this invention—in particular the increased swimming time—demonstrate that DOP treatment enhances fatigue-resistance. This swimming test is a reliable measure of anti-fatigue treatment as established in both laboratory animals and humans The present invention also shows that the positive control Rhodiola L. has anti-fatigue effects.

Stress represents the reaction of the body to stimuli that disturb its normal physiological equilibrium or homeostasis, often with detrimental effects. The weight of spleen, thymus and thyroid of the immune system are decreased by immobilized stress. Results in this invention show that the spleen index in the control group slightly decreases after the weight-loaded swimming test. Rhodiola extract increases spleen index significantly. However, for the lymphocyte proliferation assay, Rhodiola extract does not significantly increase proliferation rates of T cells and B cells compared with those of the control group.

Fatigue syndrome is a worldwide problem, with a prevalence rate of 0.4%-1%. More than 70 million people worldwide are affected by fatigue. No physical examination signs are specific to fatigue and no diagnostic tests identify this syndrome. The pathophysiological mechanism of fatigue is also unclear.

The mechanism of DOP against fatigue probably includes three aspects. One possible explanation is that DOP and Rhodiola extract could involve triglyceride (TG) (or fat) mobilization during exercise, as indicated by the decrease in TG. Energy for muscular exercise is derived initially from breakdown of muscle glycogen and later from circulating glucose released by the liver and from non-esterified fatty acids. After triglyceride mobilization, the utilization of protein and BUN levels will be decreased. Simultaneously, glucose (Glc) storage will be increased in liver and gastrocnemius. As is commonly known, glucose levels are decreased immediately after exercise, and later, non-esterified fatty acids released for circulating glucose. Such an effect might become advantageous during prolonged exercise, since better utilization of TG allows the sparing of glycogen and protein, and therefore delays fatigue.

The other possible explanation for the anti-fatigue effect of DOP is that it modifies several enzyme activities thereby preventing lipid oxidation which protects corpuscular membranes. Fatigue results in the release of reactive oxygen species (ROS) which cause lipid peroxidation of membrane structure. In fatigue condition, MDA level is increased and is accompanied by a decrease in levels of the antioxidant enzymes SOD and GSH-Px. These conditions are also marked by the release of LDH and CK into the serum, serving as an indirect index of damage to membranes. After intake of DOP and Rhodiola extract, MDA, CK, and LDH levels are decreased and SOD and GSH-Px levels are increased thereby protecting the membrane structure.

A third reason for relieving a fatigue effect of DOP is that it has an immunomodulating effect. Various studies have sought evidence for a disturbance in immunity in people with fatigue syndrome. Alteration of diverse immunological indicators, such as cytokine profile, function of natural killer cells, and responses of T cells to mitogens have been reported. The most predominant pharmacological effect of glucomannan in D. officinale is the ability to modulate immune function. Many polysaccharides have been reported to be able to activate macrophages and induce proliferation of lymphocytes, and this activation plays an important role in the immune response. In this invention, the mice of the control group show an association between physical lassitude and immunity suppression. In additional, supplementation with DOP leads to recovery of the reduced lymphocyte proliferation of chronic fatigue-challenged mice.

### INDUSTRIAL APPLICABILITY

The objective of the presently claimed invention is to provide a glucomannan with a huge molecular size of 730 kDa, called DOP, which is previously identified as the unique authentication marker of the expensive herb Dendrobium officinale, as the main component for combating fatigue in subjects in need thereof. In particular, the present invention provides the required dosage for DOP to provide a potent anti-fatigue effect, stronger than Rhodiola rosea extract, and has significant potential to form an anti-fatigue health product.

Those skilled in the art will appreciate from the foregoing description that the broad techniques of the embodiments can be implemented in a variety of forms. Therefore, while the embodiments have been described in connection with particular examples thereof, the true scope of the embodiments should not be so limited since other modifications will become apparent to the skilled practitioner upon a study of the drawings, specification, and following claims.

1. A method of alleviating body fatigue in a subject in need thereof by administrating a therapeutic amount of DOP extracted from the herb Dendrobium officinale.

2. The method according to claim 1 wherein the therapeutic amount of DOP is administered orally.

3. The method according to claim 1 wherein the DOP comprises a glucomannan with a molecular size of 730 kDa.

4. The method according to claim 1 wherein the therapeutic amount is at least 50 mg/kg administered on a daily basis.

5. The method according to claim 1 wherein the therapeutic amount is at least 4.27 mg/kg/day.

6. The method according to claim 5 wherein said subject is human

7. Use of DOP for the manufacture of a medicament for alleviating body fatigue in a subject in need thereof wherein said DOP comprises a glucomannan with a molecular size of 730 kDa extracted from the herb Dendrobium officinale.

8. The use according to claim 7 wherein said medicament is administered to said subject via oral administration.

9. The use according to claim 8 wherein said medicament is administered at a dosage of at least 50 mg/kg/day.

10. The use according to claim 8 wherein said medicament is administered at a dosage of at least 4.27 mg/kg/day.

11. The use according to claim 10 wherein said medicament is administered to a human.
